(19) United States
(12) Patent Application Publication
(10) Pub. No.: US 2016/0158414 A1
(43) Pub. Date: Jun. 9, 2016

(54) Anisotropic Constructs and Methods for Forming and Using Same for Treating Damaged Biological Tissue

(71) Applicant: CorMatrix Cardiovascular, Inc., Roswell, GA (US)

(72) Inventor: Robert G. Matheny, Norcross, GA (US)

(21) Appl. No.: 14/953,561

(22) Filed: Nov. 30, 2015

Related U.S. Application Data
(60) Provisional application No. 62/088,996, filed on Dec. 8, 2014.

(51) Int. Cl. A61L 27/36; A61L 27/54

(52) U.S. Cl. CPC A61L 27/3633; A61L 27/54; A61L 2430/20

(57) ABSTRACT

Anisotropic constructs having a base member with a defined surface that includes a plurality of substantially parallel equidistant linear channels that are configured to modulate the polarity and proliferation of cells through spatial and biomechanical cues. The constructs are also capable of administering a biologically active agent and/or a pharmacological composition to tissue.

### CROSS-REFERENCE TO RELATED PATENT APPLICATIONS

This application claims the benefit of U.S. Provisional Application No. 62/088,996, filed on Dec. 8, 2014.

### FIELD OF THE INVENTION

The present invention relates to apparatus and methods for repairing damaged or diseased tissue in mammals. More particularly, the present invention relates to anisotropic constructs for repairing and/or regenerating biological tissue, and closing openings in biological tissue.

### BACKGROUND OF THE INVENTION

Heart failure occurs in nearly 5 million people a year in the U.S. alone at a combined cost of about $40 billion annually for hospitalization and treatment of these patients. The results of all the effort and cost are disappointing with a 75% five year mortality rate for the heart failure victims. The risk factors for heart failure include high blood pressure, atrial fibrillation, valvular heart disease, and cardiomyopathy. One of the most common risk factors for heart failure is myocardial infarction (MI) or a heart attack. It is estimated that approximately 22 percent of men and 46 percent of women will develop heart failure within six years of an MI. The seminal causes of heart failure after an MI include loss of myocardium and subsequent scarring leading to impaired cardiac function.

As is well known in the art, the mammalian heart is regarded as having extremely limited regenerative capacity where only ˜5% of cardiomyocytes will regenerate under routine aging conditions every 18 months thus having a limited capacity to heal after a MI. The cardiac extracellular matrix (ECM) is critical to providing spatial and mechanical cues that modulate cardiomyocyte differentiation, polarity, migration and proliferation. The collagen network that comprises the cardiac ECM is anisotropic in nature to provide the cardiomyocytes with the optimal uni-directional polarity to withstand the stress and strain of constant contractile forces. Further, the polarity of the uni-directional polarity of the cardiomyocytes facilitates electrical signal conduction and propagation. When the anisotropic cardiac ECM is damaged due to an infarct event the cells are devoid of the spatial and mechanical cues essential to cardiac cell function.

As is also well known in the art, after a MI, the heart undergoes repair in three phases including the inflammatory phase, proliferative phase and the maturation phase. Cardiomyocyte death rapidly triggers cytokine, chemokine and adhesion molecule expression thus initiating the inflammatory phase. The inflammatory phase is an acute immune response where cytokines signal the recruitment of leukocytes, including professional phagocytes, i.e. M1 macrophages and dendritic cells that clear dead cardiomyocytes. Further, matrix metalloproteinase (MMP) expression signals the breakdown of ECM within the infarct region.

The proliferative phase is a chronic immune response where transforming growth factor beta (TGF-β) and anti-inflammatory interleukin-10 (IL-10) suppress chemokine and inflammatory cytokine response, while promoting myofibroblast cell proliferation. The myofibroblast cells then deposit a plurality of ECM proteins thus providing a “provisional” ECM.

The maturation phase is the scarring phase where the ECM proteins are crosslinked and the myofibroblast cells subsequently enter a quiescent state. The resulting accumulation of crosslinked “provisional” ECM or scar tissue often results in a disruption of the ECM network. The “provisional” ECM lacks the anisotropic network of native cardiac ECM and is structurally weaker thus detrimentally altering the ventricular geometry leading to both systolic and diastolic dysfunction.

As is further well known in the art, cardiac ECM is a complex structure comprising biochemical, bioelectrical and biomechanical signaling means. Local mechanical loading conditions and cell-ECM interactions are of vital importance to cardiac tissue engineering. A large portion of ECM is composed of anisotropically aligned collagen fibers with nano-scale diameters that significantly influence tissue architecture and electromechanical coupling. Further, micro-and/or nano-scale architecture of ECM collagen thus influence cell polarity and promote migration along collagen fibrils by providing contact guidance cues.

Normal cardiac muscle comprises an ECM foundation mediating anisotropic action potential propagation, which is required for coordination of the spatiotemporal contraction of the heart required for a sufficient ejection fraction of blood. Isotropic cardiac tissue lacks this uni-directional action potential propagation and thus, myocardium composed of isotropic tissue is unable to pump a sufficient volume of blood to maintain systemic circulation. Therefore, the isotropic cardiac tissue deposited during the maturation phase of post-MI healing often results in heart failure.

In an effort to address or remedy post-MI heart failure, medical researchers have transplanted human hematopoietic stem cells, mesenchymal stem cells, endothelial precursor cells, cardiac stem cells, and skeletal myoblasts or bone marrow cells to the myocardium, with, however, little or mixed success in satisfactory regeneration of the myocardium.

Another protocol involved injecting transforming growth factor beta preprogrammed bone marrow stem cells to the myocardium, with greater success than transplantation of bone marrow stem cells alone, but without generation of contractile myocardium.

Clinical findings also reflect that when a population of stem or progenitor cells is delivered to biological tissue, alone or on (or within) a non-linking delivery platform, i.e. merely injected into damaged tissue, a significant percentage of the cell population will either die or fail to differentiate into fully functional cardiomyocytes. Further, the limited percentage of the cell population that may differentiate into cardiomyocytes will fail to mimic the polarity of the existing cardiomyocytes that comprise the existing healthy cardiac tissue. As will be appreciated by one skilled in the art, cells grown on a planar surface having no spatial guidance cues will provide a random arrangement with no discernable alignment or polarity.

ECM based delivery platforms have also been delivered to subjects with great success in regenerating functional myocardium. The ECM delivery platforms provide the biological scaffold necessary for not only cell survival, but for cell proliferation and differentiation. Current ECM delivery platforms available have shown substantial clinical efficacy and are actively benefitting patients.

Recent studies have, however suggested that cells rely on spatial cues provided by the ECM collagen structure. While ECM delivery platforms have been found highly effective, native ECM can, and often will, provide random micro-and/or nano-scale structural cues.

Various anisotropic ECM and polymer based apparatus have also been developed in an attempt to modulate cell proliferation and polarity. Illustrative are the ECM and polymer based apparatus, i.e. grafts and endografts, disclosed in U.S. Pat. Nos. 8,613,776 and U.S. patent application Ser. Nos. 13/589,645 and 13/878,383. Biocompatible anisotropic polymers are also disclosed in Kim, et al., “Matrix Nanotopography as a Regulator of Cell Function,” Journal of Cell Biology, Vol. 197(3), pp. 351-360 (2012), Takahashi, et al., “The Use of Anisotropic Cell Sheets to Control Orientation during the Self-Organization of 3D Muscle Tissue,” Biomaterials, Vol. 43, pp. 7372-7380 (2013) and Macadangdang, et al., “Capillary Force Lithography for Cardiac Tissue Engineering,” Journal of Visualized Experiments, Vol. 88, pp. 1-8 (2014).

A major drawback of the noted polymer based apparatus, as well as most known apparatus, is that the apparatus often comprise or include a permanent structure that remains in the body, i.e. non-biodegradable. As is well known in the art, such structures (or devices) can, and in most instances will, cause irritation and undesirable biologic responses in the surrounding tissue.

Such structures (and devices) are also prone to failure, resulting in severe adverse consequences, e.g., ruptured vessels.

U.S. Pat. No. 8,613,776 discloses an ECM-based scaffold and that is fabricated in a bioreactor to produce surface features on the micro-or nano-scale. The bioreactor includes a mold configured to imprint high fidelity spatial features of the ECM-based scaffold.

A drawback of the noted scaffold includes the use of ECM-based materials, which may include collagen or basal lamina devoid of the plurality of native proteins and structures, i.e. glycosaminoglycans (GAGS), growth factors, etc., essential to tissue remodeling. Further, the scaffold is devoid of an ECM pattern that will generate an anisotropic cell growth pattern in vitro or in vivo.

U.S. patent application Ser. No. 13/589,645, entitled discloses stacked, lamellar constructs comprised of synthetic or natural polymeric membrane structures that are connected to form 3D scaffolds. The membrane structures are formed via a soft lithography means comprising a silicon master configured to provide a micro-pattern inverse of the master.

A major drawback of the noted constructs includes the use of crosslinked collagen devoid of the plurality of native proteins and structures and, hence, will not promote tissue remodeling. Further, the scaffold is devoid of an ECM pattern that will generate an anisotropic cell growth pattern in vitro or in vivo.

U.S. patent application Ser. No. 13/878,383 entitled discloses anisotropic muscle thin films (MTFs) that function as biological pacemakers and AV-bypass nodes. The MTFs comprise thin layers of ECM seeded with cardiomyocytes that were cultured in vitro. Micropatterns are imparted onto the MTFs via soft lithography, self-assembly, vapor deposition, or photolithography.

A major drawback of the noted MTF is the in vitro culturing of the cell population, which often results in phenotypic changes to the cell population that are detrimental to in vivo functionality. Additionally, the orientation or alignment of the transplanted MTF relative to the alignment of the native tissue cells will depend heavily on the skill of surgeon. Further, pre-seeding with the cells will limit the infiltration and migration of autologous cell species.

There is, thus, a need for structures or constructs that employ nanoscale patterning that effectively modulates alignment of proliferating cells during tissue remodeling to mimic the native alignment of existing tissue cells, such as grafts and vascular prostheses.

It is therefore an object of the present invention to provide anisotropic constructs having a defined, i.e. patterned, surface that is configured to modulate cell polarity and proliferation when in contact with biological tissue; particularly, damaged or diseased tissue.

It is another object of the present invention to provide anisotropic constructs that deliver one or more biologically active agents, such as cells and growth factors, and/or one or more pharmacological or therapeutic agents to biological tissue when engaged thereto.

It is another object of the present invention to provide anisotropic constructs that can be readily employed to close and maintain closure of an opening in biological tissue, e.g. a surgical incision, and, if desired, also administer one or more biologically active agents and/or one or more pharmacological or therapeutic agents to biological tissue proximate the opening when engaged to the biological tissue.

As will readily be appreciated by one having ordinary skill in the art, the anisotropic constructs of the invention provide numerous advantages over conventional apparatus for repairing and/or regenerating tissue. Among the advantages are the following:
The provision of anisotropic constructs that modulate proliferating cell alignment during tissue remodeling to mimic alignment with existing healthy tissue cells; particularly, cardiovascular tissue;
The provision of constructs that substantially reduce or eliminate (i) the harsh biological responses associated with conventional polymeric and metal ECM based and non-ECM apparatus, and (ii) the formation of inflammation and infection after deployment;
The provision of constructs that can be readily and effectively employed to treat damaged or diseased biological tissue; particularly, cardiovascular tissue;
The provision of constructs that can be readily employed to close and maintain closure of openings in biological tissue;
The provision of constructs that induce host tissue proliferation, bioremodeling and regeneration of new tissue, and tissue structures with site-specific structural and functional properties; and
The provision of constructs that effectively administer at least one biologically active agent and/or pharmacological agent or composition to a subject's tissue and, thereby produce a desired biological and/or therapeutic effect.

### SUMMARY OF THE INVENTION

The present invention is directed to anisotropic constructs for repairing and/or regenerating biological tissue and methods for forming same.

In a preferred embodiment, the anisotropic constructs comprise a planar member having at least one defined surface that is configured to modulate cell proliferation and alignment. In some embodiments, the anisotropic constructs comprise a planar member having a plurality of defined surfaces.

In some embodiments, the defined surface comprises a plurality of substantially parallel linearly grooved equidistant channels.

In some embodiments, the defined surface comprises a substantially parallel pattern having a plurality of embossed square and/or rectangular impressions.

In some embodiments, the defined surface comprises a substantially parallel pattern having a plurality of embossed circular and/or elliptical impressions.

In some embodiments, the defined surface comprises a substantially parallel pattern having a plurality of substantially parallel plurality of embossed triangular impressions.

In some embodiments, the defined surface comprises a substantially parallel pattern having a plurality of embossed polygonal impressions.

In a preferred embodiment of the invention, the planar member and, hence, anisotropic constructs forward therefrom comprise ECM derived from a mammalian tissue source selected from the group comprising, without limitation, the small intestine, large intestine, stomach, lung, liver, kidney, pancreas, placenta, heart, bladder, prostate, tissue surrounding growing enamel, tissue surrounding growing bone, and any fetal tissue from any mammalian organ. The ECM can also comprise collagen from mammalian sources.

In some embodiments of the invention, the ECM includes at least one additional biologically active agent, i.e. an agent that induces or modulates a physiological or biological process, or cellular activity, e.g., induces proliferation, and/or growth and/or regeneration of tissue.

In some embodiments of the invention, the biologically active agent comprises a cell selected from the group comprising, without limitation, embryonic stem cells, mesenchymal stem cells, hematopoietic stem cells, bone marrow stem cells, bone marrow-derived progenitor cells, myosatellite progenitor cells, totipotent stem cells, pluripotent stem cells, multipotent stem cells, oligopotent stem cells and unipotent stem cells.

In some embodiments of the invention, the biologically active agent comprises a growth factor selected from the group comprising, without limitation, transforming growth factor beta (TGF-13), fibroblast growth factor-2 (FGF-2), basic fibroblast growth factor (bFGF), vascular epithelial growth factor (VEGF) and insulin-like growth factor (IGF).

In some embodiments of the invention, the biologically active agent comprises a protein selected from the group comprising, without limitation, collagen (types I-V), proteoglycans, glycosaminoglycans (GAGs), glycoproteins, cytokines, cell-surface associated proteins, and cell adhesion molecules (CAMs).

In some embodiments of the invention, the ECM includes at least one pharmacological agent or composition, i.e. an agent, drug, compound, composition of matter or mixture thereof, including its formulation, which provides some therapeutic, often beneficial, effect.

In a preferred embodiment, the pharmacological agent or composition is selected from the group comprising, without limitation, antibiotics, anti-arrhythmic agents, anti-viral agents, analgesics, steroidal anti-inflammatories, non-steroidal anti-inflammatories, anti-neoplastics, anti-spasmodics, modulators of cell-extracellular matrix interactions, proteins, hormones, growth factors, matrix metalloproteinases (MMPs), enzymes and enzyme inhibitors, anticoagulants and/or antithrombic agents, DNA, RNA, modified DNA and RNA, NSAIDs, and inhibitors of DNA, RNA or protein synthesis.

In some embodiments of the invention, the planar member and, hence, anisotropic constructs forward therefrom comprise a biocompatible polymeric composition.

According to the invention, the polymeric composition can comprise, without limitation, polyglycolide (PGA), polylactide (PLA), poly(s-caprolactone) (PCL), poly dioxanone (a polyether-ester), poly lactide-co-glycolide, polyamide esters, polyalkalene esters, polyvinyl esters, polyvinyl alcohol, and polyanhydrides. Natural polymeric materials, include, without limitation, polysaccharides (e.g. starch and cellulose), proteins (e.g., gelatin, casein, silk, wool, etc.), and polyesters (e.g., polyhydroxyalkanoates).

According to the invention, the polymeric composition can comprise a hydrogel, without limitation, polyurethane, poly(ethylene glycol), poly(propylene glycol), poly(vinylpyrrolidone), xanthan, methyl cellulose, carboxymethyl cellulose, alginate, hyaluronan, poly(acrylic acid), polyvinyl alcohol, acrylic acid, hydroxypropyl methyl cellulose, methacrylic acid, αβ-glycerophosphate, κ-carrageenan, 2-acrylamido-2-methylpropanesulfonic acid, and β-hairpin peptide.

In some embodiments, the polymeric composition includes one of the aforementioned biologically active or pharmacological agents.

In some embodiments of the invention, the anisotropic constructs are applied as a surface layer to the leads of an implantable medical device.

According to the invention, the device can comprise, without limitation, a pacemaker and defibrillator.

In another embodiment of the invention, there is provided a method of forming the aforementioned anisotropic constructs of the invention.

### BRIEF DESCRIPTION OF THE DRAWINGS

Further features and advantages will become apparent from the following and more particular description of the preferred embodiments of the invention, as illustrated in the accompanying drawings, and in which like referenced characters generally refer to the same parts or elements throughout the views, and in which:

FIG. 1 is a perspective view of one embodiment of a planar anisotropic construct, in accordance with the invention;

FIG. 2 is a front sectional view of the anisotropic construct shown in FIG. 1, in accordance with the invention;

FIG. 3 is a top plan view of the anisotropic construct shown in FIG. 1, in accordance with the invention;

FIG. 4 is a front sectional view of another embodiment of an anisotropic construct, in accordance with the invention;

FIG. 5 is a perspective sectional view of the anisotropic construct shown in FIG. 4, in accordance with the invention;

FIG. 6 is a front sectional view of another embodiment of an anisotropic construct, in accordance with the invention;

FIG. 7 is a perspective view of a multi-layer anisotropic construct, in accordance with the invention;

FIG. 8 is a perspective sectional view of another embodiment of an anisotropic construct having the defined surface shown in FIG. 5 and polarized cell growth, in accordance with the invention;

FIG. 9 is a schematic illustration of a human heart;

FIG. 10 is a schematic illustration of the human heart shown in FIG. 9 having an infarct region;

FIG. 11 is a schematic illustration of the human heart shown in FIG. 10 having an anisotropic construct disposed over the infarct region, in accordance with the invention;

FIG. 12 is a schematic illustration of the human heart shown in FIG. 9 having a surgical incision; and

FIG. 13 is a schematic illustration of the human heart shown in FIG. 12 having an anisotropic construct disposed over the surgical incision, in accordance with the invention.

### DETAILED DESCRIPTION OF THE PREFERRED EMBODIMENT

Before describing the present invention in detail, it is to be understood that this invention is not limited to particularly exemplified apparatus, systems, materials, compositions, structures or methods as such may, of course, vary. Thus, although a number of apparatus, systems, materials, compositions, structures and methods similar or equivalent to those described herein can be used in the practice of the present invention, the preferred apparatus, systems, materials, compositions, structures and methods are described herein.

It is also to be understood that the terminology used herein is for the purpose of describing particular embodiments of the invention only and is not intended to be limiting.

Unless defined otherwise, all technical and scientific terms used herein have the same meaning as commonly understood by one having ordinary skill in the art to which the invention pertains.

Further, all publications, patents and patent applications cited herein, whether supra or infra, are hereby incorporated by reference in their entirety.

As used in this specification and the appended claims, the singular forms “a, “an” and “the” include plural referents unless the content clearly dictates otherwise. Thus, for example, reference to “an active” includes two or more such actives and the like.

Further, ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

It is also understood that there are a number of values disclosed herein, and that each value is also herein disclosed as “approximately” that particular value in addition to the value itself. For example, if the value “10” is disclosed, then “approximately 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed then “less than or equal to 10”, as well as “greater than or equal to 10” is also disclosed.

### Definitions

The terms “graft” and “prosthesis” are used interchangeably herein, and mean and include a device member or system that is configured for placement on biological tissue on or in an organ, such as a lumen or vessel. As discussed in detail herein, upon placement of a graft of the invention to biological tissue; particularly, damaged or diseased tissue the graft induces “modulated healing”.

The term “biocompatible”, as used herein, means a device or material that is substantially non-toxic in an in vivo environment, and is not substantially rejected by a recipient's physiological system, i.e. non-antigenic.

The term “anisotropic”, as used herein, means a member having physical properties or characteristics that are directionally dependent.

The term “defined surface” as used herein means and includes a surface of a structural member that is configured to provide spatial and mechanical cues that modulate cell polarity, spatial temporal positioning, differentiation, proliferation and migration when in contact with biological tissue; particularly, damaged and/or diseased tissue.

The terms “extracellular matrix” and “ECM” are used interchangeably herein, and mean and include a collagen-rich substance that is found in between cells in mammalian tissue, and any material processed therefrom, e.g. decellularized ECM. According to the invention, the ECM material can be derived from various mammalian tissue sources including, without limitation, the small intestine, large intestine, stomach, lung, liver, kidney, pancreas, placenta, heart, bladder, prostate, tissue surrounding growing enamel, tissue surrounding growing bone, and any fetal tissue from any mammalian organ.

The ECM material can thus comprise, without limitation, small intestine submucosa (SIS), urinary bladder submucosa (UBS), stomach submucosa (SS), central nervous system tissue, dermal extracellular matrix, subcutaneous extracellular matrix, gastrointestinal extracellular matrix, i.e. large and small intestines, tissue surrounding growing bone, placental extracellular matrix, ornamentum extracellular matrix, epithelium of mesodermal origin, i.e. mesothelial tissue, cardiac extracellular matrix, e.g., pericardium and/or myocardium, kidney extracellular matrix, pancreas extracellular matrix, lung extracellular matrix, and combinations thereof. The ECM can also comprise collagen from mammalian sources.

The terms “urinary bladder submucosa (UBS)”, “small intestine submucosa (SIS)” and “stomach submucosa (SS)” also mean and include any UBS and/or SIS and/or SS material that includes the tunica mucosa (which includes the transitional epithelial layer and the tunica propria), submucosal layer, one or more layers of muscularis, and adventitia (a loose connective tissue layer) associated therewith.

The ECM can also be derived from basement membrane of mammalian tissue/organs, including, without limitation, bladder, “urinary basement membrane (UBM)”, liver, i.e. “liver basement membrane (LBM)”, and amnion, chorion, allograft pericardium, allograft acellular dermis, amniotic membrane, Wharton's jelly, and combinations thereof.

Additional sources of mammalian basement membrane include, without limitation, spleen, lymph nodes, salivary glands, prostate, pancreas and other secreting glands.

The ECM can also be derived from other sources, including, without limitation, collagen from plant sources and synthesized extracellular matrices, i.e. cell cultures.

The term “angiogenesis”, as used herein, means a physiologic process involving the growth of new blood vessels from pre-existing blood vessels.

The term “neovascularization”, as used herein, means and includes the formation of functional vascular networks that can be perfused by blood or blood components. Neovascularization includes angiogenesis, budding angiogenesis, intussuceptive angiogenesis, sprouting angio genesis, therapeutic angiogenesis and vasculogenesis.

The terms “biologically active agent” and “biologically active composition” are used interchangeably herein, and mean and include agent that induces or modulates a physiological or biological process, or cellular activity, e.g., induces proliferation, and/or growth and/or regeneration of tissue.

The terms “biologically active agent” and “biologically active composition” thus mean and include, without limitation, the following growth factors: platelet derived growth factor (PDGF), epidermal growth factor (EGF), transforming growth factor alpha (TGF-α), transforming growth factor beta (TGF-β), fibroblast growth factor-2 (FGF-2), basic fibroblast growth factor (bFGF), vascular epithelial growth factor (VEGF), hepatocyte growth factor (HGF), insulin-like growth factor (IGF), nerve growth factor (NGF), platelet derived growth factor (PDGF), tumor necrosis factor alpha (TNF-α), and placental growth factor (PLGF).

The terms “biologically active agent” and “biologically active composition” also mean and include, without limitation, embryonic stem cells, mesenchymal stem cells, hematopoietic stem cells, bone marrow stem cells, bone marrow-derived progenitor cells, myosatellite progenitor cells, totipotent stem cells, pluripotent stem cells, multipotent stem cells, oligopotent stem cells and unipotent stem cells. The group also comprises cardiomyocytes, myoblasts, monocytes, parenchymal cells, epithelial cells, endothelial cells, mesothelial cells, fibroblasts, osteoblasts, chondrocytes, exogenous cells, endogenous cells, macrophages, capillary endothelial cells, xenogenic cells, allogenic cells, and cells derived from any of the three germ layers including the endoderm, mesoderm and ectoderm.

The terms “biologically active agent”.and “biologically active composition” also mean and include, without limitation, the following biologically active agents (referred to interchangeably herein as a “protein”, “peptide” and “polypeptide”): collagen (types I-V), proteoglycans, glycosaminoglycans (GAGs), glycoproteins, cytokines, cell-surface associated proteins, cell adhesion molecules (CAM), endothelial ligands, matrikines, cadherins, immuoglobins, fibril collagens, non-fibrillar collagens, basement membrane collagens, multiplexins, small-leucine rich proteoglycans, decorins, biglycans, fibromodul ins, keratocans, lumicans, epiphycans, heparin sulfate proteoglycans, perlecans, agrins, testicans, syndecans, glypicans, serglycins, selectins, lecticans, aggrecans, versicans, neurocans, brevicans, cytoplasmic domain-44 (CD-44), macrophage stimulating factors, amyloid precursor proteins, heparins, chondroitin sulfate B (dermatan sulfate), chondroitin sulfate A, heparin sulfates, hyaluronic acids, fibronectins, tenascins, elastins, fibrillins, laminins, nidogen/enactins, fibulin I, fibulin II, integrins, transmembrane molecules, thrombospondins, ostepontins, and angiotensin converting enzymes (ACE).

The terms “pharmacological agent”, “active agent”, “drug” and “active agent formulation” are used interchangeably herein, and mean and include an agent, drug, compound, composition of matter or mixture thereof, including its formulation, which provides some therapeutic, often beneficial, effect. This includes any physiologically or pharmacologically active substance that produces a localized or systemic effect or effects in animals, including warm blooded mammals, humans and primates; avians; domestic household or farm animals, such as cats, dogs, sheep, goats, cattle, horses and pigs; laboratory animals, such as mice, rats and guinea pigs; fish; reptiles; zoo and wild animals; and the like.

The terms “pharmacological agent”, “active agent”, “drug” and “active agent formulation” thus mean and include, without limitation, antibiotics, anti-arrhythmic agents, anti-viral agents, analgesics, steroidal anti-inflammatories, non-steroidal anti-inflammatories, anti-neoplastics, anti-spasmodics, modulators of cell-extracellular matrix interactions, proteins, hormones, growth factors, matrix metalloproteinases (MMPs), enzymes and enzyme inhibitors, anticoagulants and/or antithrombic agents, DNA, RNA, modified DNA and RNA, NSAIDs, inhibitors of DNA, RNA or protein synthesis, polypeptides, oligonucleotides, polynucleotides, nucleoproteins, compounds modulating cell migration, compounds modulating proliferation and growth of tissue, and vasodilating agents.

The terms “pharmacological agent”, “active agent”, “drug” and “active agent formulation” thus include, without limitation, atropine, tropicamide, dexamethasone, dexamethasone phosphate, betamethasone, betamethasone phosphate, prednisolone, triamcinolone, triamcinolone acetonide, fluocinolone acetonide, anecortave acetate, budesonide, cyclosporine, FK-506, rapamycin, ruboxistaurin, midostaurin, flurbiprofen, suprofen, ketoprofen, diclofenac, ketorolac, nepafenac, lidocaine, neomycin, polymyxin b, bacitracin, gramicidin, gentamicin, oyxtetracycline, ciprofloxacin, ofloxacin, tobramycin, amikacin, vancomycin, cefazolin, ticarcillin, chloramphenicol, miconazole, itraconazole, trifluridine, vidarabine, ganciclovir, acyclovir, cidofovir, ara-amp, foscarnet, idoxuridine, adefovir dipivoxil, methotrexate, carboplatin, phenylephrine, epinephrine, dipivefrin, timolol, 6-hydroxydopamine, betaxolol, pilocarpine, carbachol, physostigmine, demecarium, dorzolamide, brinzolamide, latanoprost, sodium hyaluronate, insulin, verteporfin, pegaptanib, ranibizumab, and other antibodies, antineoplastics, anti-VEGFs, ciliary neurotrophic factor, brain-derived neurotrophic factor, bFGF, Caspase-1 inhibitors, Caspase-3 inhibitors, α-Adrenoceptors agonists, NMDA antagonists, Glial cell line-derived neurotrophic factors (GDNF), pigment epithelium-derived factor (PEDF), and NT-3, NT-4, NGF, IGF-2.

The terms “pharmacological agent”, “active agent”, “drug” and “active agent formulation” further mean and include the following Class I—Class V anti-arrhythmic agents: (Class Ia) quinidine, procainamide and disopyramide; (Class Ib) lidocaine, phenytoin and mexiletine; (Class Tc) flecainide, propafenone and moricizine; (Class II) propranolol, esmolol, timolol, metoprolol and atenolol; (Class III) amiodarone, sotalol, ibutilide and dofetilide; (Class IV) verapamil and diltiazem) and (Class V) adenosine and digoxin.

The terms “pharmacological agent”, “active agent”, “drug” and “active agent formulation” further mean and include, without limitation, the following antibiotics: aminoglycosides, cephalosporins, chloramphenicol, clindamycin, erythromycins, fluoroquinolones, macrolides, azolides, metronidazole, penicillins, tetracyclines, trimethoprim-sulfamethoxazole and vancomycin.

The terms “pharmacological agent”, “active agent”, “drug” and “active agent formulation” further include, without limitation, the following steroids: andranes (e.g., testosterone), cholestanes, cholic acids, corticosteroids (e.g., dexamethasone), estraenes (e.g., estradiol) and pregnanes (e.g., progesterone).

The terms “pharmacological agent”, “active agent”, “drug” and “active agent formulation” can further include one or more classes of narcotic analgesics, including, without limitation, morphine, codeine, heroin, hydromorphone, levorphanol, meperidine, methadone, oxycodone, propoxyphene, fentanyl, methadone, naloxone, buprenorphine, butorphanol, nalbuphine and pentazocine.

The terms “pharmacological agent”, “active agent”, “drug” and “active agent formulation” can further include one or more classes of topical or local anesthetics, including, without limitation, esters, such as benzocaine, chloroprocaine, cocaine, cyclomethycaine, dimethocaine/larocaine, piperocaine, propoxycaine, procaine/novacaine, proparacaine, and tetracaine/amethocaine. Local anesthetics can also include, without limitation, amides, such as articaine, bupivacaine, cinchocaine/dibucaine, etidocaine, levobupivacaine, lidocaine/lignocaine, mepivacaine, prilocaine, ropivacaine, and trimecaine. Local anesthetics can further include combinations of the above from either amides or esters.

The terms “anti-inflammatory” and “anti-inflammatory agent” are also used interchangeably herein, and mean and include a “pharmacological agent” and/or “active agent formulation”, which, when a therapeutically effective amount is administered to a subject, prevents or treats bodily tissue inflammation i.e. the protective tissue response to injury or destruction of tissues, which serves to destroy, dilute, or wall off both the injurious agent and the injured tissues.

Anti-inflammatory agents thus include, without limitation, alclofenac, alclometasone dipropionate, algestone acetonide, alpha amylase, amcinafal, amcinafide, amfenac sodium, amiprilose hydrochloride, anakinra, anirolac, anitrazafen, apazone, balsalazide disodium, bendazac, benoxaprofen, benzydamine hydrochloride, bromelains, broperamole, budesonide, carprofen, cicloprofen, cintazone, cliprofen, clobetasol propionate, clobetasone butyrate, clopirac, cloticasone propionate, cormethasone acetate, cortodoxone, decanoate, deflazacort, delatestryl, depo-testosterone, desonide, desoximetasone, dexamethasone dipropionate, diclofenac potassium, diclofenac sodium, diflorasone diacetate, cliflumidone sodium, diflunisal, difluprednate, diftalone, dimethyl sulfoxide, drocinonide, endrysone, enlimomab, enolicam sodium, epirizole, etodolac, etofenamate, felbinac, ifenamole, fenbufen, fenclofenac, fenclorac, fendosal, fenpipalone, fentiazac, flazalone, fluazacort, flufenamic acid, flumizole, flunisolide acetate, flunixin, flunixin meglumine, fluocortin butyl, fluorometholone acetate, fluquazone, flurbiprofen, fluretofen, fluticasone propionate, furaprofen, furobufen, halcinonide, halobetasol propionate, halopredone acetate, ibufenac, ibuprofen, ibuprofen aluminum, ibuprofen piconol, ilonidap, indomethacin, indomethacin sodium, indoprofen, indoxole, intrazole, isoflupredone acetate, isoxepac, isoxicam, ketoprofen, lofemizole hydrochloride, lomoxicam, loteprednol etabonate, meclofenamate sodium, meclofenamic acid, meclorisone dibutyrate, mefenamic acid, mesalamine, meseclazone, mesterolone, methandrostenolone, methenolone, methenolone acetate, methylprednisolone suleptanate, momiflumate, nabumetone, nandrolone, naproxen, naproxen sodium, naproxol, nimazone, olsalazine sodium, orgotein, orpanoxin, oxandrolane, oxaprozin, oxyphenbutazone, oxymetholone, paranyline hydrochloride, pentosan polysulfate sodium, phenbutazone sodium glycerate, pirfenidone, piroxicam, piroxicam cinnamate, piroxicam olamine, pirprofen, prednazate, prifelone, prodolic acid, proquazone, proxazole, proxazole citrate, rimexolone, romazarit, salcolex, salnacedin, salsalate, sanguinarium chloride, seclazone, sermetacin, stanozolol, sudoxicam, sulindac, suprofen, talmetacin, talniflumate, talosalate, tebufelone, tenidap, tenidap sodium, tenoxicam, tesicam, tesimide, testosterone, testosterone blends, tetrydamine, tiopinac, tixocortol pivalate, tolmetin, tolmetin sodium, triclonide, triflumidate, zidometacin, and zomepirac sodium.

The term “pharmacological composition”, as used herein, means and includes a composition comprising a “pharmacological agent” and/or a “biologically active agent” and/or any additional agent or component identified herein.

The term “ECM composition”, as used herein, means and includes a composition comprising at least one ECM.

The term “therapeutically effective”, as used herein, means that the amount of the “pharmacological composition” and/or “pharmacological agent” and/or “biologically active agent” administered is of sufficient quantity to ameliorate one or more causes, symptoms, or sequelae of a disease or disorder. Such amelioration only requires a reduction or alteration, not necessarily elimination, of the cause, symptom, or sequelae of a disease or disorder.

The terms “prevent” and “preventing” are used interchangeably herein, and mean and include reducing the frequency or severity of a disease or condition. The term does not require an absolute preclusion of the disease or condition. Rather, this term includes decreasing the chance for disease occurrence.

The terms “treat” and “treatment” are used interchangeably herein, and mean and include medical management of a patient with the intent to cure, ameliorate, stabilize, or prevent a disease, pathological condition, or disorder. The terms include “active treatment”, i.e. treatment directed specifically toward the improvement of a disease, pathological condition, or disorder, and “causal treatment”, i.e. treatment directed toward removal of the cause of the associated disease, pathological condition, or disorder.

The terms “treat” and “treatment” further include “palliative treatment”, i.e. treatment designed for the relief of symptoms rather than the curing of the disease, pathological condition, or disorder, “preventative treatment”, i.e. treatment directed to minimizing or partially or completely inhibiting the development of the associated disease, pathological condition, or disorder, and “supportive treatment”, i.e. treatment employed to supplement another specific therapy directed toward the improvement of the associated disease, pathological condition, or disorder.

The terms “optional” and “optionally” mean that the subsequently described event, circumstance, or material may or may not occur or be present, and that the description includes instances where the event, circumstance, or material occurs or is present and instances where it does not occur or is not present.

The terms “patient” and “subject” are used interchangeably herein, and mean and include warm blooded mammals, humans and primates; avians; domestic household or farm animals, such as cats, dogs, sheep, goats, cattle, horses and pigs; laboratory animals, such as mice, rats and guinea pigs; fish; reptiles; zoo and wild animals; and the like.

The term “comprise” and variations of the term, such as “comprising” and “comprises,” means “including, but not limited to” and is not intended to exclude, for example, other additives, components, integers or steps.

The following disclosure is provided to further explain in an enabling fashion the best modes of performing one or more embodiments of the present invention. The disclosure is further offered to enhance an understanding and appreciation for the inventive principles and advantages thereof, rather than to limit in any manner the invention. The invention is defined solely by the appended claims including any amendments made during the pendency of this application and all equivalents of those claims as issued.

As discussed above, the present invention is directed to anisotropic constructs for repairing damaged or diseased tissue and/or regenerating biological tissue and methods for forming same. As will readily be appreciated by one having ordinary skill in the art, the present invention substantially reduces or eliminates the disadvantages and drawbacks associated with prior art methods and apparatus for repairing damaged or diseased tissue, and closing openings in biological tissue.

As indicated above, in a preferred embodiment, the anisotropic constructs comprise a planar member having at least one defined surface that is configured to modulate cell proliferation and alignment. In some embodiments, the constructs comprise a planar member having a plurality of defined surfaces.

According to the invention, the defined surface(s) induce and/or modulate cell polarity and proliferation when in contact with biological tissue; particularly, damaged or diseased tissue.

in some embodiments of the invention, the defined surface comprises a plurality of substantially parallel linearly grooved equidistant channels.

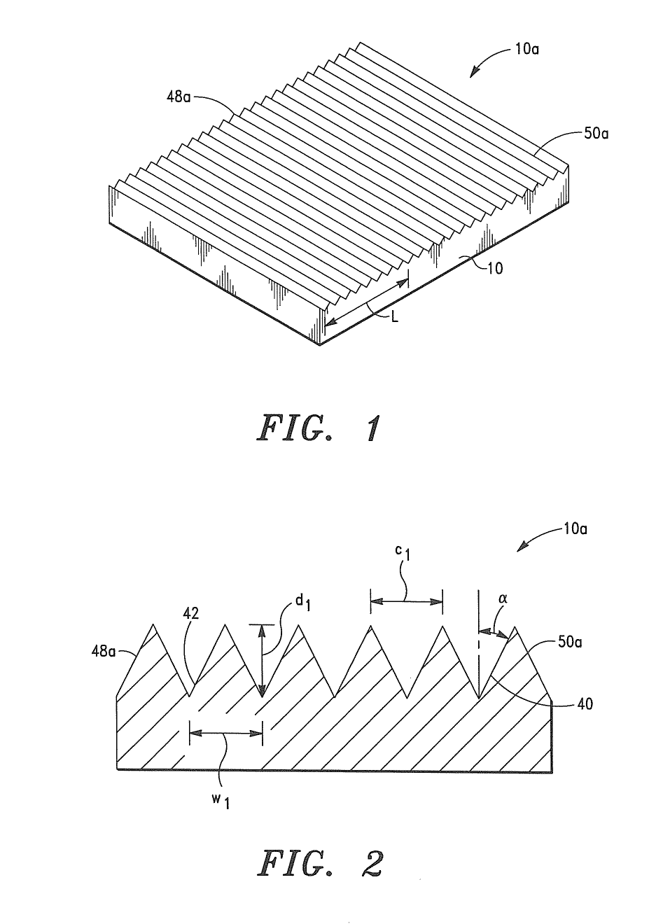

In a preferred embodiment, the defined surface comprises a channel density (“σ”); channel density being defined herein as the number of channels per linear length (measured perpendicular to the channels), i.e. line “L” in FIG. 1.

In a preferred embodiment of the invention, the defined surfaces of the anisotropic constructs have a channel density “σ” in the range of approximately 1-5000 channels/mm, more preferably, in the range of approximately 100-1000 channels/mm.

In some embodiments, the defined surface comprises a plurality of parallel embossed triangular impressions having a plurality of channels therebetween.

As discussed in detail herein, in a preferred embodiment, the base width (“W₁”) of each triangular impression is in the range of approximately 0.1-100 μm. (See FIG. 2)

In a preferred embodiment, the channel width (“C₁”) proximate the surface of each triangular impression is in the range of approximately 0.1-100 μm and depth (“d₁”) of the corresponding channels is in the range of approximately 0.1-100 μm.

In a preferred embodiment, each face of a triangular impression is oriented at an angle (“α”) in the range of 0-90° relative to a line corresponding to the plane defined by a channel (perpendicular to the horizontal of the defined surface).

In some embodiments, the defined surface comprises a plurality of substantially parallel embossed square and/or rectangular impressions having a plurality of channels therebetween.

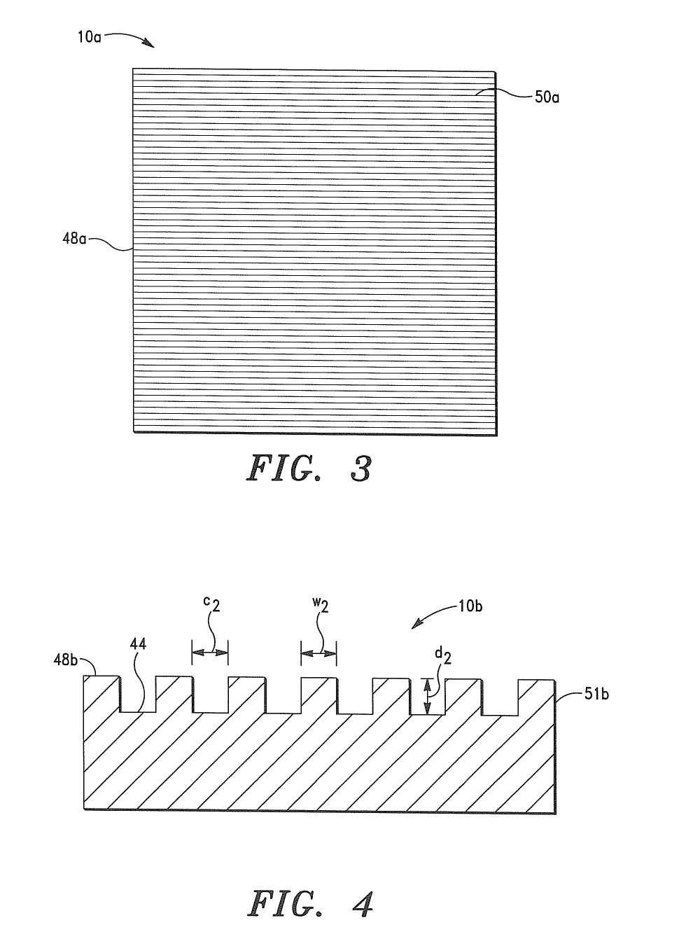

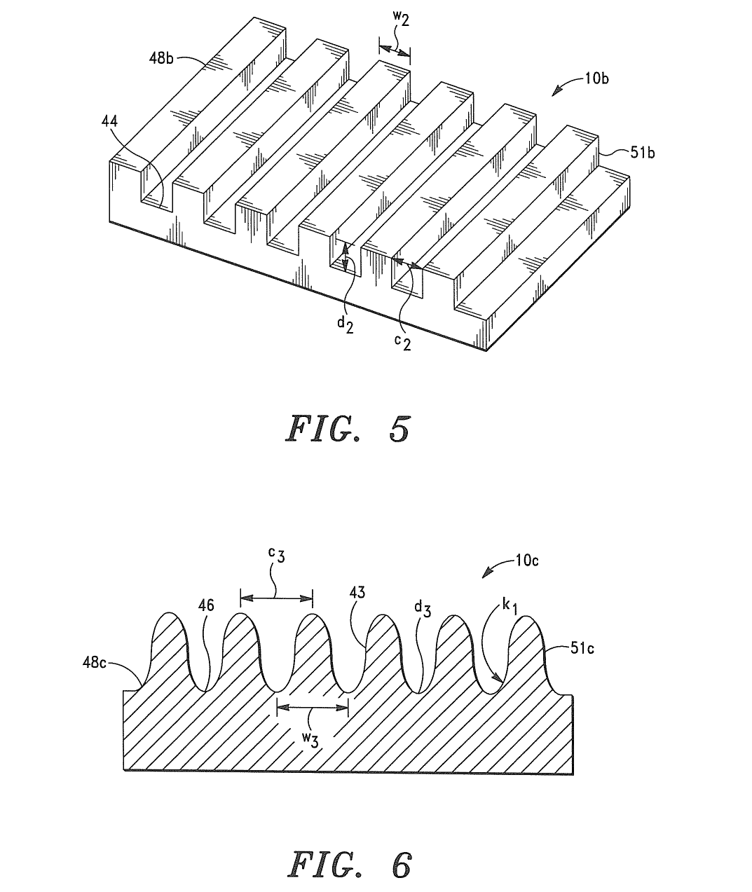

In a preferred embodiment, the width (“W₂”) of each raised square configuration is in the range of approximately 0.1-100 μm. (See FIG. 4)

As discussed herein, in a preferred embodiment, the width (“C₂”) of the channel between each raised square configuration and depth (“d₂”) of the channels is in the range of approximately 0.1-100 μm.

In some embodiments, the defined surface comprises a plurality of substantially parallel embossed circular and/or elliptical impressions having a plurality of channels therebetween.

As discussed herein, in a preferred embodiment, the circular and/or elliptical impressions comprise an elongated elliptical shape, i.e. linear or curvilinear sides and a circular top.

In a preferred embodiment, the base width (“W₃”) of each circular and/or elliptical impression is in the range of 0.1-100 μm. (See FIG. 6)

In a preferred embodiment, the channel width (“C₁”) proximate the surface defined by of the circular and/or elliptical impressions is in the range of approximately 0.1-100 μm and depth (“d₁”) of the corresponding channels is in the range of approximately 0.1-100 μm.

In a preferred embodiment, the channel width (“C₃”) between adjacent peaks of the circular and/or elliptical impressions is in the range of approximately 0.1 μm-300 mm and the depth (“d₃”) of the channels is in the range of approximately 0.1-100 μm.

In some embodiments, the defined surface comprises a plurality of pores having a diameter in the range of approximately 0.1 μm-1 mm.

As indicated above, in a preferred embodiment of the invention, the planar member and, hence, anisotropic constructs forward therefrom comprises ECM derived from a mammalian tissue source. According to the invention, the ECM can be derived from various mammalian tissue sources and methods for preparing same, such as disclosed in U.S. Pat. Nos. 7,550,004, 7,244,444, 6,379,710, 6,358,284, 6,206,931, 5,733,337 and 4,902,508 and U.S. patent application Ser. No. 12/707,427; which are incorporated by reference herein in their entirety.

In a preferred embodiment of the invention, the construct ECM is derived from various mammalian tissue sources including, without limitation, the small intestine, large intestine, stomach, lung, liver, kidney, pancreas, placenta, heart, bladder, prostate, tissue surrounding growing enamel, tissue surrounding growing bone, and any fetal tissue from any mammalian organ. The ECM can also comprise collagen from mammalian sources.

According to the invention, the mammalian tissue can thus comprise, without limitation, small intestine submucosa (SIS), urinary bladder submucosa (UBS), stomach submucosa (SS), central nervous system tissue, epithelium of mesodermal origin, i.e. mesothelial tissue, dermal extracellular matrix, subcutaneous extracellular matrix, gastrointestinal extracellular matrix, i.e. large and small intestines, tissue surrounding growing bone, placental extracellular matrix, omentum extracellular matrix, cardiac extracellular matrix, e.g., pericardium and/or myocardium, kidney extracellular matrix, pancreas extracellular matrix, lung extracellular matrix, and combinations thereof. The ECM can also comprise collagen from mammalian sources.

The ECM can also be derived from the same or different mammalian tissue sources, as disclosed in Co-Pending U.S. patent application Ser. Nos. 13/033,053 and 13/033,102; which are incorporated by reference herein.

According to the invention, the ECM can also be sterilized via applicant's proprietary novasterilis process disclosed in Co-Pending U.S. patent application Ser. No. 13/480,205; which is expressly incorporated by reference herein in its entirety.

As stated above, in some embodiments of the invention, the ECM compositions and/or materials and, hence, anisotropic ECM constructs formed therefrom, include at least one additional biologically active agent or composition, i.e. an agent that induces or modulates a physiological or biological process, or cellular activity, e.g., induces proliferation, and/or growth and/or regeneration of tissue.

Suitable biologically active agents include any of the aforementioned biologically active agents, including, without limitation, the aforementioned cells, proteins and growth factors.

In some embodiments, the ECM compositions and/or materials and, hence, anisotropic constructs form therefrom, include at least one pharmacological agent or composition (or drug), i.e. an agent or composition that is capable of producing a desired biological effect in vivo, e,g., stimulation or suppression of apoptosis, stimulation or suppression of an immune response, etc.

Suitable pharmacological agents and compositions include any of the aforementioned agents, including, without limitation, antibiotics, anti-viral agents, analgesics, steroidal anti-inflammatories, non-steroidal anti-inflammatories, anti-neoplastics, anti-spasmodics, modulators of cell-extracellular matrix interactions, proteins, hormones, enzymes and enzyme inhibitors, anticoagulants and/or anti-thrombic agents, DNA, RNA, modified DNA and RNA, NSAIDs, inhibitors of DNA, RNA or protein synthesis, polypeptides, oligonucleotides, polynucleotides, nucleoproteins, compounds modulating cell migration, compounds modulating proliferation and growth of tissue, and vasodilating agents.

In some embodiments of the invention, the pharmacological agent comprises a statin, i.e. a HMG-CoA reductase inhibitor. According to the invention, suitable statins include, without limitation, atorvastatin (Lipitor®), cerivastatin, fluvastatin (Lescol®), lovastatin (Mevacor®. Altocor®, Altoprev®), mevastatin, pitavastatin (Livalo ®, Pitava®), pravastatin (Pravachol®, Selektine®, Lipostat®), rosuvastatin (Crestor®), and simvastatin (Zocor® Lipex®). Several actives comprising a combination of a statin and another agent, such as ezetimbe/simvastatin (Vytorin®), are also suitable.

Applicant has found that the noted statins exhibit numerous beneficial properties that provide several beneficial biochemical actions or activities. The properties and beneficial actions are set forth in Applicant's Co-Pending U.S. patent application Ser. No. 13/373,569, filed on Sep. 24, 2012 and Ser. No. 13/782,024, filed on Mar. 1, 2013; which are incorporated by reference herein in their entirety.

Additional suitable pharmacological agents and compositions that can be delivered within the scope of the invention are disclosed in Pat. Pub. Nos. 20070014874, 20070014873, 20070014872, 20070014871, 20070014870, 20070014869, and 20070014868; which are expressly incorporated by reference herein in its entirety.

According to the invention, the biologically active and pharmacological agents referenced above can comprise various forms. In some embodiments of the invention, the biologically active and pharmacological agents, e.g. simvastatin, comprise microcapsules that provide delayed delivery of the agent contained therein.

In some embodiments of the invention, the biologically active agent comprises a protein selected from the group comprising, without limitation, collagen (types I-V), proteoglycans, glycosaminoglycans (GAGs), glycoproteins, cytokines, cell-surface associated proteins, and cell adhesion molecules (CAMs).

In some embodiments of the invention, the ECM constructs comprise a percent volume of GAGs between 0.001-20% providing a Young's modulus in the range of 30-1000 KPa.

In some embodiments, the ECM planar member comprises a sheet of ECM (and/or a biologically active and/or pharmacological agent augmented ECM sheet). In some embodiments, the ECM planar member comprises a plurality of the aforementioned ECM sheets.

According to the invention, upon deployment of an anisotropic construct of the invention to damaged and/or diseased biological tissue, “modulated healing” is effectuated.

The term “modulated healing”, as used herein, and variants of this language generally refer to the modulation (e.g., alteration, delay, retardation, reduction, etc.) of a process involving different cascades or sequences of naturally occurring tissue repair in response to localized tissue damage or injury, substantially reducing their inflammatory effect. Modulated healing, as used herein, includes many different biologic processes, including epithelial growth, fibrin deposition, platelet activation and attachment, inhibition, proliferation and/or differentiation, connective fibrous tissue production and function, angiogenesis, and several stages of acute and/or chronic inflammation, and their interplay with each other.

For example, in some embodiments, the ECM constructs of the invention are specifically formulated (or designed) to alter, delay, retard, reduce, and/or detain one or more of the phases associated with healing of damaged tissue, including, but not limited to, the inflammatory phase (e.g., platelet or fibrin deposition), and the proliferative phase when in contact with biological tissue.

In some embodiments, when the ECM construct is in contact with biological tissue modulated healing is effectuated through the direct replacement of cardiac ECM, which promotes the quiescence of myofibroblasts, thus preventing the deposit of fibrotic isotropic ECM. Further, the defined surface of the ECM construct promotes infiltration and spatial temporal alignment of contractile cardiomyocytes to provide functional cardiac tissue.

In some embodiments, “modulated healing” refers to the ability of an ECM construct to alter a substantial inflammatory phase (e.g., platelet or fibrin deposition) at the beginning of the tissue healing process. As used herein, the phrase “alter a substantial inflammatory phase” refers to the ability of an ECM construct to substantially reduce the inflammatory response at an injury site when in contact with biological tissue.

In such an instance, a minor amount of inflammation may ensue in response to tissue injury, but this level of inflammation response, e.g., platelet and/or fibrin deposition, is substantially reduced when compared to inflammation that takes place in the absence of an ECM construct of the invention.

For example, the ECM constructs discussed herein have been shown experimentally to delay or alter the inflammatory response associated with damaged tissue, as well as excessive formation of connective fibrous tissue following tissue damage or injury. The ECM constructs have also been shown experimentally to delay or reduce fibrin deposition and platelet attachment to a blood contact surface following tissue damage.

In some embodiments of the invention, “modulated healing” refers to the ability of an ECM construct of the invention to induce host tissue proliferation, bioremodeling, including neovascularization, e.g., vasculogenesis, angiogenesis, and intussusception, and regeneration of tissue structures with site-specific structural and functional properties.

Accordingly, the ECM constructs of the invention provide an excellent means for treating damaged or diseased biologically tissue, including closing and maintaining closure of openings in biological tissue, e.g., closure of openings in tissue after surgical intervention.

In some embodiments of the invention, the planar member and, hence, anisotropic constructs therefrom comprise a biocompatible polymeric composition.

According to the invention, the polymeric composition can comprise, without limitation, polyglycolide (PGA), polylactide (PLA), poly(r-caprolactone) (PCL), poly dioxanone (a polyether-ester), poly lactide-co-glycolide, polyamide esters, polyalkalene esters, polyvinyl esters, polyvinyl alcohol, and polyanhydrides. Natural polymeric materials, include, without limitation, polysaccharides (e.g. starch and cellulose), proteins (e.g., gelatin, casein, silk, wool, etc.), and polyesters (e.g., polyhydroxyalkanoates).

According to the invention, the polymeric composition can comprise a hydrogel, without limitation, polyurethane, poly(ethylene glycol), poly(propylene glycol), poly(vinylpyrrolidone), xanthan, methyl cellulose, carboxymethyl cellulose, alginate, hyaluronan, poly(acrylic acid), polyvinyl alcohol, acrylic acid, hydroxypropyl methyl cellulose, methacrylic acid, αβ-glycerophosphate, κ-carrageenan, 2-acrylamido-2-methylpropanesulfonic acid, and 3-hairpin peptide.

In some embodiments, the hydrogel is crosslinked via chemically and/or photocuring, e.g. ultraviolet light.

As indicated above, in some embodiments, the polymeric composition includes one of the aforementioned biologically active or pharmacological agents.

Thus, in some embodiments of the invention, when a polymeric anisotropic construct of the invention is administered to damaged and/or diseased biological tissue, “modulated healing” is similarly effectuated.

According to the invention, various conventional methods can be employed to form the aforementioned ECM constructs of the invention, including, without limitation, microfabrication manufacturing means including soft lithography, i.e. Polydimethylsiloxane (PDMS) and/or silicon molds, photolithography, chemical etching, enzymatic etching, deep reactive ion etching (DRIE), reactive ion etching (RIE), focused ion beam (FIB) etching, wet etching, bulk micromachining, surface micromachining, laser micromachining, and/or X-ray lithography, electroplating, and molding (AGA) techniques on or to a glass, semiconductor, or polymer substrate.

In some embodiments, the soft lithography PDMS and/or silicon mold comprises, without limitation, a stamp, roller, block, tile, embossing device, or imprinting device.

in some embodiments, the enzymatic etching process comprises a controlled exposure to collagenases including, but not limited to dispase, AUX-I, AUX-II, matrix metalloprotease-1 (MMP-1), matrix metalloprotease-8 (MMP-8) and matrix metalloprotease-13 (MMP-13).

Referring now to FIGS. 1-3, there is shown one embodiment of an anisotropic construct 10a of the invention. As illustrated in FIG. 1, the construct 10a comprises a base member 10 having a defined surface 48a. In this embodiment, the defined surface 48a comprises a plurality of substantially parallel triangular equidistant members 50a having channels 42 therebetween.

According to the invention, the base member 10 can comprise one of the aforementioned ECM or polymeric materials.

As indicated above and illustrated in FIG. 2, each triangular member 50a comprises a depth (“d₁”) and base width (“W₁”) in the range of 0.1-100 μm.

In a preferred embodiment, each face 40 of a triangular member 50a is oriented at an angle (“α”) in the range of 0-90° relative to a line corresponding to the plane defined by a channel 42.

Referring now to FIGS. 4 and 5, there is shown another embodiment of anisotropic construct 10b of the invention. As illustrated in FIG. 5, the construct 10b comprises a base member 10 having defined surface 48b. In this embodiment, the defined surface 48b comprises a plurality of substantially parallel square and/or rectangular equidistant members 51b having a plurality of corresponding square and/or rectangular channels 44 therebetween.

According to the invention, the base member 10 can similarly comprise one of the aforementioned ECM or polymeric materials.

As illustrated in FIG. 4, each member 51b comprises a depth (“d₂”) and width (“W₂”) in the range of approximately 0.1-100 μm. The channels 44 between each member 51b have a width (‘C₂) in the range of approximately 0.1-100 μm.

Referring now to FIG. 6, there is shown another embodiment of anisotropic construct 10c of the invention. As illustrated in FIG. 6, the construct 10c comprises a base member 10 having a defined surface 48c. In this embodiment, the defined surface 48c comprises a plurality of substantially parallel circular and/or elliptical equidistant members 51c. In a preferred embodiment, the members 51c have an elongated elliptical shape, i.e. two linear or curvilinear sides and a circular top.

According to the invention, the base member 10 can similarly comprise one of the aforementioned ECM or polymeric materials.

In a preferred embodiment, the base width (“W₃”) of each circular and/or elliptical impression is in the range of 0.1-100 μm.

In a preferred embodiment, the channel width (“C₃”) proximate the surface defined by of the circular and/or elliptical impressions is in the range of approximately 0.1-300 mm. In a preferred embodiment, the depth (“d₃”) of the corresponding channels is similarly in the range of approximately 0.1 μm-300 mm.

In a preferred embodiment, each circular and/or elliptical member 51c has a mean curvature k₁. As will be appreciated by one having ordinary skill in the art, k₁ will vary as a function of member width (“W₃”).

According to the invention, the anisotropic constructs 50a, 50b, 50c can be formed with various desired pre-deployment shapes, e.g. concave or convex, to facilitate contact, preferably, substantially full contact to host tissue.

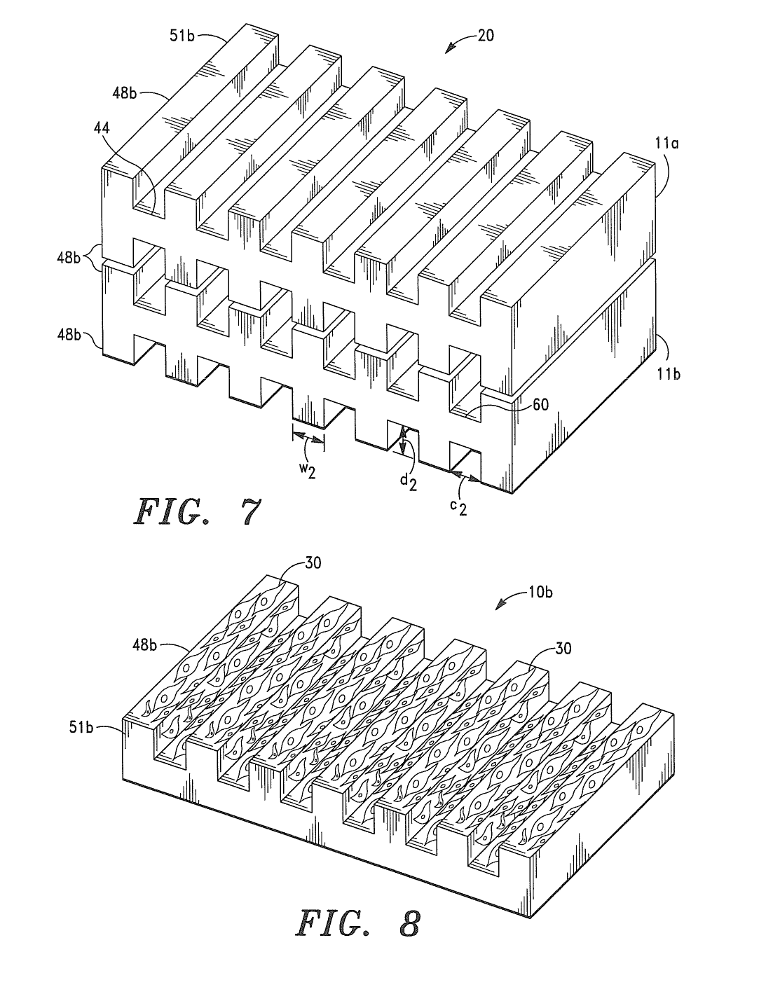

Referring now to FIG. 7, there is shown another embodiment of an anisotropic construct of the invention. As illustrated in FIG. 7, the anisotropic construct 20 comprises a plurality of base members 1la, 11b, i.e. a multi-member construct.

According to the invention, the base members 11a, 11b can similarly comprise one of the aforementioned ECM or polymeric materials.

In some embodiments, the base members 11a, 11b include top and bottom defined surfaces 48b, i.e. a plurality of substantially parallel equidistant square and/or rectangular canals 51b.

As illustrated in FIG. 7, the base members are in communication with each other and are oriented wherein a plurality of openings 60 are provided.

According to the invention, the multi-member anisotropic construct 20 can comprise more than two members, e.g. 3, 4, 5 members.

Referring now to FIG. 8, there is shown anisotropic construct 10b having a plurality of polarized cells 30 aligned with the substantially parallel equidistant square and/or rectangular canals 51b.

According to the invention, cells 30 can comprise, without limitation, embryonic stem cells, mesenchymal stem cells, hematopoietic stem cells, bone marrow stem cells, bone marrow-derived progenitor cells, myosatellite progenitor cells, totipotent stem cells, pluripotent stem cells, multipotent stem cells, oligopotent stem cells and unipotent stem cells.

According to the invention, the polarized cells 30 can be provided [or induced] by various conventional means, including, without limitation, chemotaxis (migration), differentiation and/or proliferation of local in vivo cell populations, seeding and/or injecting with autologous and/or allogeneic cells cultured in vitro.

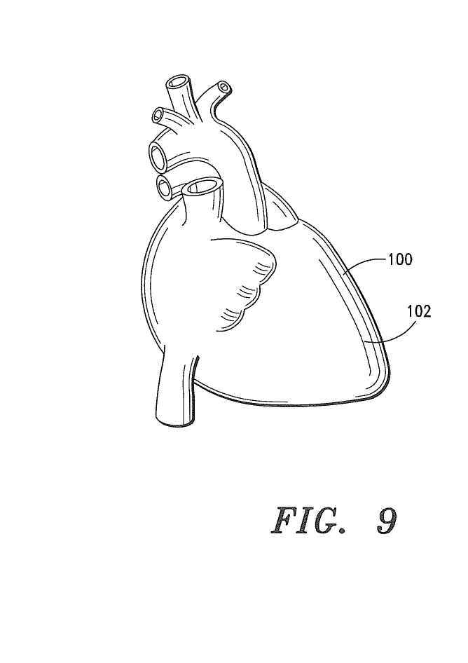

Referring now to FIG. 9 there is shown a depiction of a normal heart 100. As is well known in the art, the heart wall 102 consists of an inner layer of simple squamous epithelium, referred to as the endocardium. The endocardium overlays the myocardium (a variably thick heart muscle) and is enveloped within a multi-layer tissue structure referred to as the pericardium.

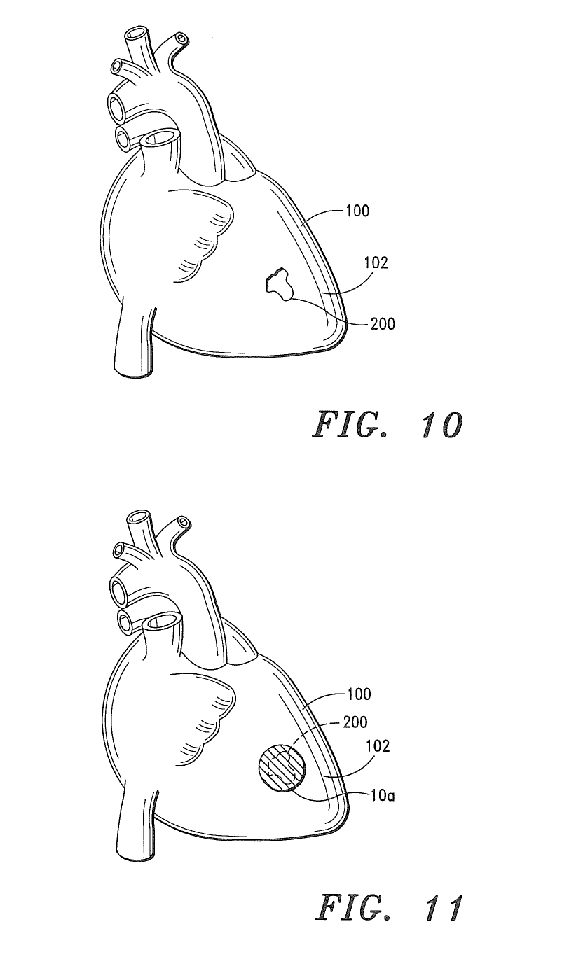

Referring now to FIG. 10, there is shown a depiction of the heart 100 with an ischemic infracted region 200. According to the invention, the infarcted region 200 can be effectively treated by disposing an anisotropic construct of the invention, e.g., anisotropic construct 10a, proximate, more preferably, directly over the infarcted region 200, as shown in FIG. 11.

As indicated above, the anisotropic constructs of the invention can also be readily employed to close openings in biological tissue; openings resulting from tissue damage or disease and/or openings resulting from surgical intervention.

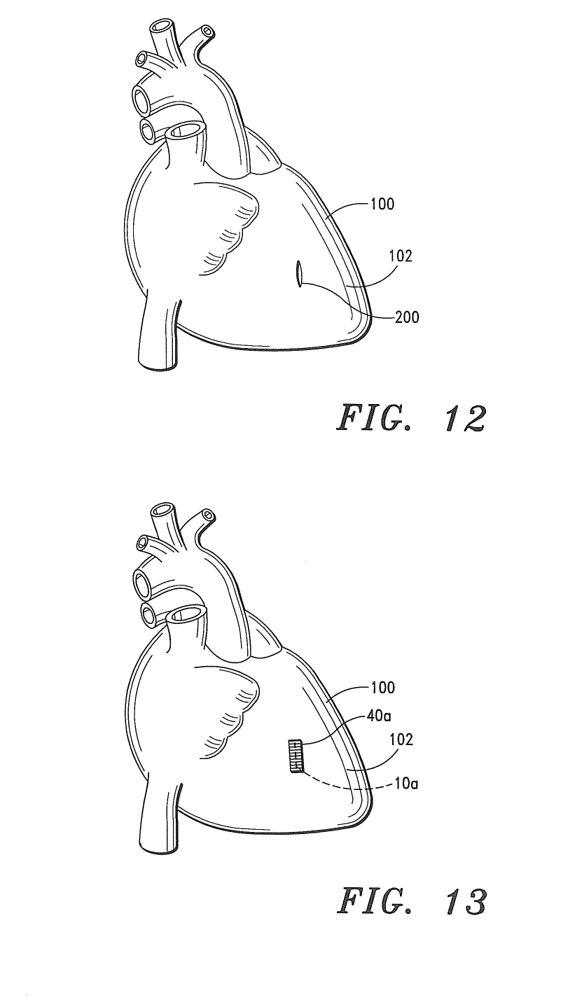

Referring now to FIG. 12, there is shown a depiction of the heart 100 with a surgical incision 202. Referring to FIG. 13, there is shown an anisotropic construct of the invention, e.g. anisotropic construct 10a, disposed over the “now closed” incision 202.

As indicated above, in addition to effectively closing the incision 202, the anisotropic construct will also induce modulated healing.

Further, if the anisotropic construct includes a pharmacological agent (or composition), a desired therapeutic action is also effectuated.

One having ordinary skill in the art will thus readily appreciate that the anisotropic constructs of the invention provide numerous advantages over conventional apparatus for repairing and/or regenerating tissue. Among the advantages are the following:
The provision of anisotropic constructs that can be readily and effectively employed to treat damaged or diseased biological tissue; particularly, cardiovascular tissue;
The provision of anisotropic constructs that can be readily employed to close and maintain closure of openings in biological tissue;
The provision of anisotropic constructs having defined surface layer configured to provide spatial and mechanical cues that modulate cell polarity, spatial temporal positioning, differentiation, proliferation and migration when in contact with biological tissue; particularly, damaged and/or diseased tissue cells;
The provision of anisotropic constructs that induce modulated healing thus modulating the immune response to an infarct event;
The provision of anisotropic constructs that substantially reduce deposition of “provisional” fibrotic ECM associated with the post-infarct immune response;
The provision of anisotropic constructs that induce host tissue proliferation, bioremodeling and regeneration of new tissue, and tissue structures with site-specific structural and functional properties;
The provision of anisotropic constructs that provide remodeled cardiac tissue having a tensile strength substantially similar to native tissue;
The provision of anisotropic constructs that substantially reduce or eliminate (i) the harsh biological responses associated with conventional polymeric and metal apparatus, and (ii) the formation of inflammation and infection after deployment; and
The provision of anisotropic constructs that effectively administer at least one biologically active agent and/or pharmacological agent or composition to a subject's tissue to induce a desired biological and/or therapeutic effect.

A further advantage of the anisotropic constructs of the invention is that they can be readily employed in various medical procedures, including, without limitation, treatment of coronary and peripheral vascular disease (PVD) in cardiovascular vessels, including, but not limited to, iliacs, superficial femoral artery, renal artery, tibial artery, popliteal artery, etc., deep vein thromboses (DVT), vascular bypasses, and coronary vascular repair.

The anisotropic constructs of the invention of the invention can also be readily incorporated in or employed with various cardiovascular conduits, valves and grafts, including, without limitation, the heart valves, conduits and grafts disclosed in U.S. Pat. No. 7,998,196 and U.S. patent appllication Ser. No. 13/782,024, filed Mar. 1, 2013, Ser. No. 13/782,289, filed Mar. 1, 2013, Ser. No. 13/804,683, filed Mar. 14, 2013 and Ser. No. 13/328,287, filed Dec. 16, 2011.

It is envisioned that the anisotropic constructs of the invention of the invention can also be readily incorporated in or employed with a microneedle structure, including, without limitation the biodegradeable support scaffolds disclosed in U.S. Pat. No. 8,778,012, which is incorporated by reference herein.

Without departing from the spirit and scope of this invention, one of ordinary skill can make various changes and modifications to the invention to adapt it to various usages and conditions. As such, these changes and modifications are properly, equitably, and intended to be, within the full range of equivalence of any issued claims.

1. An anisotropic construct for repairing damaged biological tissue, comprising:
a planar member comprising an extracellular matrix (ECM) composition, said ECM composition comprising acellular ECM from a mammalian tissue source,
said planar member comprising at least one defined outer surface, said at least on defined outer surface comprising a nanoscale surface,
said planar member being configured to induce modulated healing when administered to damaged biological tissue, said modulated healing comprising modulation of inflammation and induced bioremodeling and regeneration of tissue structures with site-specific structural and functional properties,
wherein, during said bioremodeling, said at least one defined surface modulates cell proliferation, polarity and alignment.

2. The anisotropic construct of claim 1, wherein said at least one defined surface comprises a plurality of substantially parallel linearly grooved equidistant channels.

3. The anisotropic construct of claim 1, wherein said at least one defined surface comprises a plurality of embossed rectangular impressions.

4. The anisotropic construct of claim 1, wherein said tissue source is selected from the group consisting of the small intestine, large intestine, stomach, lung, liver, kidney, pancreas, placenta, heart, bladder, prostate, tissue surrounding growing enamel, tissue surrounding growing bone, and fetal tissue from a mammalian organ.

5. The anisotropic construct of claim 4, wherein said tissue source comprises an adolescent mammalian tissue source.

6. The anisotropic construct of claim 1, wherein said ECM composition comprises at least one supplemental biologically active agent.

7. The anisotropic construct of claim 6, wherein said at least one supplemental biologically active agent comprises a cell selected from the group consisting of an embryonic stem cell, mesenchymal stem cell, hematopoietic stem cell, bone marrow stem cell and, bone marrow-derived progenitor cell and myosatellite progenitor cell.

8. The anisotropic construct of claim 6, wherein said at least one supplemental biologically active agent comprises a growth factor selected from the group consisting of transforming growth factor alpha (TGF-α), transforming growth factor beta (TGF-β), fibroblast growth factor-2 (FGF-2), basic fibroblast growth factor (bFGF), vascular epithelial growth factor (VEGF), and insulin-like growth factor (IGF).

9. The anisotropic construct of claim 6, wherein said at least one supplemental biologically active agent comprises a protein selected from the group consisting of collagen (types I-V), proteoglycans, glycosaminoglycans (GAGs), glycoproteins, cytokines, cell-surface associated proteins, and cell adhesion molecules (CAMs).

10. The anisotropic construct of claim 9, wherein said planar member comprises in the range of 0.001-20% vol. of said GAGs and a Young's modulus in the range of 30-1000 KPa.

11. The anisotropic construct of claim 1, wherein said ECM composition comprises at least one pharmacological agent.

12. The anisotropic construct of claim 11, wherein said pharmacological agent comprises an anti-inflammatory selected from the group consisting of steroidal anti-inflammatories and non-steroidal anti-inflammatories.

13. The anisotropic construct of claim 11, wherein said pharmacological agent comprises a statin selected from the group consisting of atorvastatin, cerivastatin, fluvastatin, lovastatin, mevastatin, pitavastatin, pravastatin, rosuvastatin, and simvastatin.

14. The anisotropic construct of claim 1, wherein said planar member further comprises a biodegradeable support scaffold comprising a microneedle structure.
